(19) United States
(12) Patent Application Publication
(10) Pub. No.: US 2022/0112257 A1
(43) Pub. Date: Apr. 14, 2022

(54) MUL TISPECIFIC PROCHEMOKINE THERAPEUTIC PROTEINS (PARK) AND METHOD OF MAKING AND USING THEREOF

(71) Applicant: ARBELE LIMITED, Shatin, N.T., Hong Kong (CN)

(72) Inventor: Donald E. STAUTON, Kirkland, WA (US)

(21) Appl. No.: 17/422,095

(22) PCT Filed: Jan. 11, 2020
(86) PCT No.: PCT/US2020/013257

§ 371 Date: Jul. 9, 2021

Related U.S. Application Data
(60) Provisional application No. 62/791,677, filed on Jan. 11, 2019.

(51) Int. Cl. C07K 14/52; C07K 16/28; A61K 45/06; A61K 38/19; A61K 47/68

(52) U.S. Cl. CPC C07K 14/523; C07K 16/2896; A61K 45/06; A61K 38/195; C07K 2317/622; C07K 2319/33; C07K 2319/30; C07K 2319/21; C07K 2319/22; A61K 47/6813

(57) ABSTRACT

A ProteAse Released chemoKines protein (PARK) comprises a prochemokine moiety comprising a propeptide moiety fused to a chemokine moiety, wherein the chemokine moiety comprises a N-terminus and a C-terminus; and a targeting moiety linked to the prochemokine moiety, wherein the targeting moiety has a binding specificity to a tumor, fibrosis or Alzheimer's Disease associated antigen or receptor.

### CROSS REFERENCE TO RELATED APPLICATION

This claims the benefit of the filing date of U.S. Provisional Ser. No. 62/791,667 filed Jan. 11, 2019 under 35 U.S.C. 119(e), the entire disclosure of which is incorporated by reference herein.

### TECHNICAL FIELD

The present application generally relates to the technical field of recombinant proteins and antibodies, and more particularly relates to therapeutic proteins and antibodies to treat cancer, fibrotic and neurodegenerative diseases.

### BACKGROUND

Many studies have shown that low levels of tumor lymphocyte infiltration are correlated with poor prognosis (Jung 2014, Zhang 2003, Naito 1998). Enhancing tumor lymphocyte infiltration increases an anti-tumor immune response and decreases tumor growth. For example, treatment with chemokines can result in complete tumor regression or no tumor growth, indicating that chemokine administration alone or in combination with a cytokine can be therapeutic (Homey 2002). CCL21 is a C—C chemokine ligand, also known as Secondary Lymphoid-tissue Chemokine (SLC) and can elicit its effects by binding to a chemokine receptor, CCR7. CCL21 is expressed by and secreted from the fibroblastic reticular cells as a chemoattractant to guide naive, CCR7-expressing T cells to the T cell zone within human lymph nodes. It has been demonstrated that SLC chemokine injection into a hepatocellular carcinoma (HCC) model increases CD8 and CD4 positive T cell populations at the tumor site (Chen 2013). SLC induces the maturation of dendritic cells, increases levels of IL-12 and IFNγ, and inhibits HCC growth and invasiveness. Other studies show that reversal of tumor epigenetic silencing of TH1 chemokines, CXCL9 and CXCL10, both of which are CXC chemokines, increases tumor lymphocyte infiltration, slows down tumor progression, and improves the efficacy of PD-L1 blockade (Peng 2015).

In many cancer patients, there are evidences of inadequate anti-tumor immune response, and low levels of immune cells infiltrating in a tumor is indicative of poor prognosis. The underlying mechanism may be related to poor stimulation and/or suppression of tumor cell initiated immune response. These observations present an opportunity to improve the efficacy of cancer treatment by increasing immune cells at the tumor site.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

The disclosure provides generally therapeutic proteins, nucleic acid sequences or expression vectors encoding such proteins or fragments thereof, cells for expressing such proteins or fragments thereof, methods of making such proteins and methods of treating disease using such proteins.

In one aspect, the disclosure provides therapeutic proteins. The protein may be useful for treating diseases including cancer, fibrosis, or Alzheimer's Disease. In one embodiment, the protein comprises a prochemokine moiety and a targeting moiety linked to the prochemokine moiety.

The prochemokine moiety comprises a propeptide moiety fused to a chemokine moiety, and the chemokine moiety comprises a N-terminus and a C-terminus. In one embodiment, the propeptide moiety is fused to either the N-terminus or the C-terminus of the chemokine moiety. In one embodiment, the propeptide moiety is fused to the N-terminus of the chemokine moiety. In one embodiment, the propeptide moiety comprises from about 5 to about 20 amino acids. In one embodiment, the propeptide moiety comprises about 8 amino acids.

The targeting moiety has the binding specificity to a tumor, fibrosis or Alzheimer's Disease associated antigen or receptor.

In one embodiment, the prochemokine moiety further comprises a leader, and the propeptide moiety is fused to the leader at one end and to chemokine moiety at the other end. In one embodiment, the leader comprises an amino acid sequence having from about 10 to about 50 amino acids. In one embodiment, the leader comprises an amino acid sequence having from about 15 to about 25 amino acids.

In one embodiment, the protein contains a single prochemokine moiety. In one embodiment the protein contains two or more prochemokine moieties. In one embodiment, the protein comprises at least two prochemokine moieties. In one embodiment, the two prochemokine moieties are linked in tandem through a spacer. In one embodiment, the spacer comprises at least one amino acid residue. In one embodiment, the amino acid residue comprises a glycine residue. In one embodiment, the spacer comprises at least two glycine residues. In one embodiment, the spacer comprises 2 to 8 glycine residues.

In one embodiment, the protein further comprises an amino acid sequence fused at the C-terminus of the chemokine moiety, wherein the amino acid sequence comprises the mucin region of fractalkine.

In one embodiment, the chemokine moiety comprises a chemokine amino acid sequence having at least 70%, 75%, 80%, 85%, 90%, 95%, 97%, 98%, 99% or 100% similarity to CC, CXC, CX3C, OR C family chemokines. In one embodiment, the similarity is 90%. In one embodiment, the similarity is 98%. In one embodiment, the similarity is 99%.

In one embodiment, the chemokine moiety comprises a chemokine amino acid sequence having at least 70%, 75%, 80%, 85%, 90%, 95%, 97%, 98%, 99% or 100% similarity to CCL1, CCL3, CCL5, CCL7, CCL14, CCL16, CCL19, CCL20, CCL21, CXCL8, CXCL9, CXCL10, CXCL12, CXCL16, XCL1, or CX3CL1. In one embodiment, the similarity is 90%. In one embodiment, the similarity is 98%. In one embodiment, the similarity is 99%.

In one embodiment, the chemokine moiety comprises a chemokine amino acid sequence having at least 70%, 75%, 80%, 85%, 90%, 95%, 97%, 98%, 99% or 100% similarity to a chemokine capable of recruiting cells with anti-tumor, anti-fibrosis or anti-Alzheimer's Disease activity. In one embodiment, the similarity is 90%. In one embodiment, the similarity is 98%. In one embodiment, the similarity is 99%.

In one embodiment, the targeting moiety comprises an antibody, a single chain Fv (scFv) domain, a single variable domain or a natural ligand domain that has the binding affinity to a tumor, fibrosis or Alzheimer's Disease associated antigen or receptor. In one embodiment, the K_{d} of the binding is not more than 1, 5, 10, 15, 20, 25, 30, 50, 60, 80, 90, 100, 150 nM. In one embodiment, the targeting moiety is configured to bind a tumor cell, tumor stroma antigen, myofibroblast antigen, amyloid or tau protein.

In one embodiment, the protein further comprises a protein tag. In one embodiment, the protein tag is convalently linked to one of the proteins. In one embodiment, the protein tag comprises an amino acid sequence having at least 70%, 75%, 80%, 85%, 90%, 95%, 97%, 98%, 99% or 100% similarity to an amino acid sequence comprising from about 6 to about 10 histidine residues, streptag-2 or immunoglobulin Fc. In one embodiment, the similarity is 90%. In one embodiment, the similarity is 98%. In one embodiment, the similarity is 99%.

In one embodiment, the protein further comprises an immunoglobulin (IgG) Fc domain. In one embodiment, both the prochemokine moiety and the targeting moiety are fused to the IgG Fc domain. In one embodiment, the targeting moiety is fused onto one end of the prochemokine moiety and the IgG Fc is fused onto the opposite end of the prohemokine domain. In one embodiment, the IgG Fc domain comprises two immunoglobulin CH2-CH3 domains. In one embodiment, one of the immunoglobulin CH2-CH3 domain is fused to the targeting moiety and the other immunoglobulin CH2-CH3 domain is fused to the prochemokine moiety. In one embodiment, the protein comprises at least two prochemokine moieties.

In one embodiment, the targeting moiety comprises a scFv domain. In one embodiment, the protein comprises one scFv domain and one prochemokine moiety. In one embodiment, the protein comprises at least two scFv domains.

In one embodiment, the protein is an antibody. In one embodiment, the antibody is a bispecific antibody.

In a second aspect, the disclosure provides isolated nucleic acid sequence encoding the therapeutic proteins or a fragment thereof disclosed herein. In one embodiment, the disclosure provides an isolated nucleic acid encoding a leader, a prochemokine moiety and a tumor, fibrosis or Alzheimer's Disease-targeting moiety.

In a further aspect, the disclosure provides an expression vector comprising the isolated nucleic acid disclosed thereof. In one embodiment, the vector is expressible in a cell.

In a further aspect, the disclosure provides a host cell. In one embodiment, the host cell comprises the nucleic acid disclosed herein. In one embodiment, the host cell comprises the expression vector disclosed herein. In one embodiment, the host cell is a prokaryotic cell. In one embodiment, the host cell is a eukaryotic cell. In one embodiment, the host cell is a mammalian cell, a yeast, or a bacterium.

In a further aspect, the disclosure provides methods of producing proteins or a fragment thereof disclosed herein. In one embodiment, the method comprises culturing a host cell so that the desired protein is produced.

In a further aspect, the disclosure provides pharmaceutical compositions. In one embodiment, the composition is useful for treating cancers, fibrosis or Alzheimer's disease. In one embodiment, the pharmaceutical composition comprises the protein disclosed herein and a pharmaceutically acceptable carrier. In one embodiment, the composition further comprises radioisotope, radionuclide, a toxin, a therapeutic agent, a chemotherapeutic agent or a combination thereof.

In a further aspect, the disclosure provides methods for treating a subject with a cancer, fibrosis or Alzheimer's Disease. In one embodiment, the method comprises administering to the subject an effective amount of the protein disclosed herein.

In one embodiment, the cancer comprises a solid or hematologic tumor. In one embodiment, the cancer is one selected from the group consisting of breast cancer, colorectal cancer, pancreatic cancer, head and neck cancer, liver cancer, melanoma, ovarian cancer, prostate cancer, lung cell cancer, leukemia, lymphoma, or multiple myeloma.

In one embodiment, the tumor is capable of expressing one or more tumor associated proteases, wherein the tumor associated protease(s) is capable of cleaving the prochemokine moiety off the protein and releasing an active chemokine. In one embodiment, the active chemokine released from the protein is capable of recruiting immune cells to stimulate therapeutic activity in immune or other cell types within a tumor, fibrosis or Alzheimer's Disease lesion.

In one embodiment, the fibrosis comprises kidney, liver, lung or cardiac fibrosis.

In one embodiment, the method of treatment further comprises co-administering an effective amount of a therapeutic agent. In one embodiment, the therapeutic agent comprises an antibody, a chemotherapy agent, an enzyme, or a combination thereof. In one embodiment, the therapeutic agent comprises an anti-estrogen agent, a receptor tyrosine inhibitor, an anti-fibrotic drug, an anti-Alzheimer's Disease drug or a combination thereof. In one embodiment, the therapeutic agent comprises capecitabine, cisplatin, trastuzumab, fulvestrant, tamoxifen, letrozole, exemestane, anastrozole, aminoglutethimide, testolactone, vorozole, formestane, fadrozole, letrozole, erlotinib, lafatinib, dasatinib, gefitinib, imatinib, pazopinib, lapatinib, sunitinib, nilotinib, sorafenib, nab-palitaxel.

In one embodiment, the subject is a human.

In a further aspect, the disclosure provides a solution comprising an effective concentration of the protein disclosed herein. In one embodiment, the solution is blood plasma in a subject.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 demonstrates (A) an example of converting a chemokine, RANTES, to a novel prochemokine, ProRANTES, by fusing the leader sequence of CCL14 to the sequences of a propeptide and the active portion of RANTES; and (B) the sequence of an example of prochemokines in Pro-CCL21-XCL1;

FIG. 2 shows assays for identifying prochemokines specifically cleaved by disease associated proteases, such as tumor-associated proteases;

FIG. 3 displays examples of prochemokine modified antibodies (PARK) and the protease cleavage sites;

FIG. 4 shows examples of PARK expression vectors coexpressing a PARK protein containing an Fc;

FIG. 5 shows examples of PARK proteins for treating cancer, fibrosis, and Alzheimer's disease (AD), respectively; and

FIG. 6 elucidates that a PARK platform can be used as therapeutics to recruit different cytotoxic cells against a tumor and activate innate, acquired, and/or engineered anti-tumor responses.

### DETAILED DESCRIPTION

In the following detailed description, reference is made to the literature references (which are incorporated herein by reference in their entirety) and accompanying drawings (which form a part hereof). In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as commonly understood by those of ordinary skill in the art to which the present disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

As used herein, the terms “function” and “functional” and the like refer to a biological, binding, or therapeutic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

“Homology” refers to the percentage number of amino acids that are identical or constitute conservative substitutions. Homology may be determined using sequence comparison programs such as GAP (Deveraux et al., 1984, Nucleic Acids Research 12, 387-395) which is incorporated herein by reference. In this way sequences of a similar or substantially different length to those cited herein could be compared by insertion of gaps into the alignment, such gaps being determined, for example, by the comparison algorithm used by GAP.

The term “host cell” includes an individual cell or cell culture which can be or has been a recipient of any recombinant vector(s) or isolated polynucleotide of the present disclosure. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the present disclosure. A host cell which comprises a recombinant vector of the present disclosure is a recombinant host cell.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The recitation “polynucleotide” or “nucleic acid” as used herein designates mRNA, RNA, cRNA, rRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA and RNA.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs that encode these enzymes.

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes”, which typically catalyze (i.e., increase the rate of) various chemical reactions.

The recitation polypeptide “variant” refers to polypeptides that are distinguished from a reference polypeptide sequence by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The term “reference sequence” refers generally to a nucleic acid coding sequence, or amino acid sequence, to which another sequence is being compared. All polypeptide and polynucleotide sequences described herein are included as references sequences.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99%, or 100% sequence identity to any of the reference sequences described herein (see, e.g., Sequence Listing), typically where the polypeptide variant maintains at least one biological activity of the reference polypeptide.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95%, 96%, 97%, 98%, 99% or greater of some given quantity.

“Treating” or “treatment” or “alleviation” refers to both therapeutic treatment and prophylactic or preventative measures, wherein the object is to prevent or slow down (lessen) the targeted pathologic condition or disorder. For example, for cancer, reduction in the number of cancer cells or absence of the cancer cells; reduction in the tumor size; inhibition (i.e., slow to some extent and preferably stop) of tumor metastasis; inhibition, to some extent, of tumor growth; increase in length of remission, and/or relief to some extent, one or more of the symptoms associated with the specific cancer; reduced morbidity and mortality, and improvement in quality of life issues. Reduction of the signs or symptoms of a disease may also be felt by the patient. Treatment can achieve a complete response, defined as disappearance of all signs of cancer, or a partial response, wherein the size of the tumor is decreased, preferably by more than 50 percent, more preferably by 75%. A patient is also considered treated if the patient experiences stable disease. In a preferred embodiment, the cancer patients are still progression-free in the cancer after one year, preferably after 15 months. These parameters for assessing successful treatment and improvement in the disease are readily measurable by routine procedures familiar to a physician of appropriate skill in the art.

The terms “modulating” and “altering” include “increasing” and “enhancing” as well as “decreasing” or “reducing,” typically in a statistically significant or a physiologically significant amount or degree relative to a control. In specific embodiments, immunological rejection associated with transplantation of the blood substitutes is decreased relative to an unmodified or differently modified stem cell by at least 10%, at least 15%, at least 20%, at least 25%, at least 30%, at least 40%, at least 50%, at least 60%, at least 70%, at least 80%, at least 90%, at least 100%, at least 150%, at least 200%, at least 300%, at least 400%, at least 500%, or at least 1000%.

An “increased” or “enhanced” amount is typically a “statistically significant” amount, and may include an increase that is 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

By “obtained from” is meant that a sample such as, for example, a polynucleotide or polypeptide is isolated from, or derived from, a particular source, such as a desired organism or a specific tissue within a desired organism. “Obtained from” can also refer to the situation in which a polynucleotide or polypeptide sequence is isolated from, or derived from, a particular organism or tissue within an organism. For example, a polynucleotide sequence encoding a reference polypeptide described herein may be isolated from a variety of prokaryotic or eukaryotic organisms, or from particular tissues or cells within certain eukaryotic organism. A “therapeutically effective amount” refers to an amount of an antibody or a drug effective to “treat” a disease or disorder in a subject. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See preceding definition of “treating”.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Vector” includes shuttle and expression vectors. Typically, the plasmid construct will also include an origin of replication (e.g., the ColE1 origin of replication) and a selectable marker (e.g., ampicillin or tetracycline resistance), for replication and selection, respectively, of the plasmids in bacteria. An “expression vector” refers to a vector that contains the necessary control sequences or regulatory elements for expression of the antibodies including antibody fragment of the present disclosure, in bacterial or eukaryotic cells. Suitable vectors are disclosed below.

The term “antibody” is used in the broadest sense and specifically covers monoclonal antibodies (including full length monoclonal antibodies), multi-specific antibodies (e.g., bispecific antibodies), and antibody fragments so long as they exhibit the desired biological activity or function.

“Antibody fragments” comprise a portion of a full-length antibody, generally the antigen binding or variable region of the antibody. Examples of antibody fragments include Fab, Fab′, F(ab′)2, and Fv fragments; diabodies; linear antibodies; single-chain antibody molecules; and multi-specific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three complementarity determining regions (CDRs) specific for an antigen) has the ability to recognize and bind antigen, although at a lower affinity than the entire binding site.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The term “variable” refers to the fact that certain segments of the variable domains (V domains) differ extensively in sequence among antibodies. The V domain mediates antigen binding and defines specificity of a particular antibody for its particular antigen. However, the variability is not evenly distributed across the 10-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four frameworks regions (FRs), largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a CDR (e.g. around about residues 24-34 (L1), 50-56 (L2) and 89-97 (L3) in the VL, and around about 31-35B (H1), 50-65 (H2) and 95-102 (H3) in the VH (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the VL, and 26-32 (H1), 52A-55 (H2) and 96-101 (H3) in the VH (Chothia and Lesk J. Mol. Biol. 196:901-917 (1987)).

“Chimeric” antibodies (immunoglobulins) have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. Proc. Natl Acad. Sci. USA 81:6851-6855 (1984)). Humanized antibody as used herein is a subset of chimeric antibodies.

“Humanized” forms of non-human (e.g., murine) antibodies are chimeric antibodies which contain minimal sequence derived from non-human immunoglobulin. In some embodiments, humanized antibodies are human immunoglobulins (recipient or acceptor antibody) in which hypervariable region residues of the recipient are replaced by hypervariable region residues from a non-human species (donor antibody) such as mouse, rat, rabbit or nonhuman primate having the desired specificity, affinity, and capacity. In some embodiments, humanized antibodies are antibodies derived from human cells or from transgenic animals (typically mice) with express human antibody genes.

The present disclosure relates to therapeutic protein, and method of making and using thereof for inducing or enhancing an innate, acquired, or engineered (chimeric antigen receptor mediated), anti-tumor immune response by increasing the recruitment and activation of immune cells at tumor sites, especially those tumors often consisting low levels of lymphocyte infiltration.

In one aspect, the disclosure provides therapeutic proteins. The protein may be antibodies. The proteins may be configured to release chemokine at the therapeutic site, which attract immune cells to the therapeutic site.

In one embodiment, the disclosure provides multifunctional recombinant proteins having binding affinity to tumor cells or tumor stroma where bioactive chemokines are released to attract different classes of lymphocytes, monocytes, dendritic cells, NK cells, or chimeric antigen receptor (CAR) expressing cells to the tumor. Specifically, a recombinant protein may be manufactured as a therapeutic product comprising at least one prochemokine moiety and at least one tumor-targeting moiety for binding to a tumor or tumor stroma associated protein or antigen. The tumor-targeting moiety may be an antibody, a single-chain variable fragment (scFv), a variable heavy single domain i.e. a nanobody or a natural ligand domain that specifically binds to a tumor associated antigen or receptor. When the recombinant protein is administered to a cancer patient, the recombinant protein recognizes and binds to the tumor cells or within the tumor microenvironment. As in many cases of solid tumors, the tumor or tumor stroma associated protease activity may cleave the prochemokine of the recombinant protein at the site of tumor to release the chemokine. Then the free and bioactive chemokine is capable of recruiting immune cells to the site of tumor. The bioactive chemokine may also stimulate immune cells resulting in an anti-tumor response. Such a recombinant protein is a therapeutic protein designated as “PARK” for ProteAse Released chemoKines.

In one embodiment, the disclosure provides multifunctional PARK proteins having binding affinity to cells or proteins that are associated with fibrosis or Alzheimer's disease. In one embodiment, PARK may be targeted to fibroblast activation protein alpha (FAPa) expressed on myofibroblasts in fibrosis or amyloid beta (A13) in Alzheimer's disease. Proteases active at these sites such as uPA may release bioactive chemokines that possess appropriate protease substrate sequences. The released chemokines may demonstrate therapeutic activity. For example, CXCL9 and CXCL10 may decrease hepatic stellate cell collagen production in liver fibrosis (Liang 2012). CXCL12 may decrease neuronal apoptosis induced by Aβ (Zuena 2019).

In one embodiment, the assays are provided for screening and identifying propeptides that are specifically cleaved by at least one tumor associated protease. The objectives and advantages of the present disclosure may become apparent from the following detailed description of preferred embodiments in examples thereof in connection with the accompanying drawings.

### EXAMPLES

### Example 1: Converting a Chemokine to a Novel Prochemokine

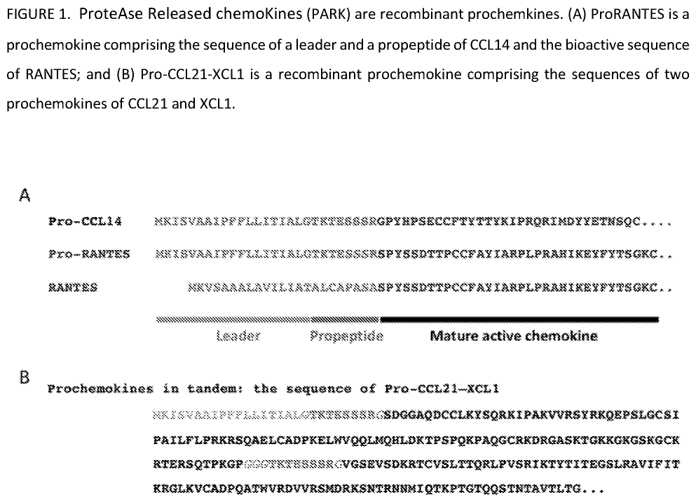

The chemokine, CCL14 (a.k.a. Hemofiltrate CC chemokine, as described in Detheux 2000, Vakili 2001, Blain 2007), is normally present in blood. An eight-amino acid propeptide is proteolytically cleaved from the N-terminus of CCL14 for activation of CCL14 to bind to its receptors (SEQ ID NO: 1-4). Propeptide cleavage activates potent binding to chemokine receptors CCR1, CCR3, and CCR5, and subsequently, T cell and monocyte chemotaxis (Detheux 2000, Vakili 2001, Blain 2007). Based on this biochemical feature of CCL14, other chemokines are converted to prochemokines enabling the construction of PARK proteins with a range of specific cell activation and recruitment to tumors. As shown in FIG. 1, RANTES (SEQ ID NO: 5-7), also known as CCL5, is chemotactic for T cells, eosinophils, and basophils, and plays an active role in recruiting leukocytes into inflammatory sites, as well as in suppressing HIV infection. As shown in FIG. 1, RANTES is converted into ProRANTES (SEQ ID NO: 8) by fusing the leader and propetide sequence of CCL14 (SEQ ID NO: 2-3) to the bioactive portion of RANTES (SEQ ID NO: 7, FIG. 1A). Additional PARK is generated by fusing the leader and propetide sequence of CCL14 to individual bioactive chemokine sequences (SEQ ID NO: 11-20), which result in the formation of corresponding prochemokines with their SEQ ID NO: 111-201. Prochemokine-in-tandem is generated by inserting an additional propeptide to each additional bioactive chemokine sequences, such as the example of CCL21-XCL1 prochemokine (SEQ ID NO: 211) as shown in FIG. 1B. A spacer sequence of one to four glycine may be inserted N-terminus to the propetide to increase efficiency of proteolytic cleavage. With certain chemokines a single glycine may be inserted c-terminal to the propeptide to increase proteolytic cleavage. Chemokines is also converted to prochemokines by inserting a propeptide between the C-terminus of their leader peptide and the N-terminus of the mature active form of the chemokine. Various leader and propeptide sequences may be used to create novel prochemokines (Table 1). Propeptides may be selected from a database of propeptides (http://www.cbs.dtu.dk/databases/propeptides/). Protease substrate sequences may be selected from a database of cleavage sites http://pmap.burnharm.org/proteases).

### Example 2. Identification of Tumor-Associated Proteases and PARK Prochemokines

Certain chemokines are stable and active as monomers, such as CCL21 (SLC) or CCL7 (MCP3). Other chemokines may form dimers that enable binding to glycosylaminolycans and support haptotaxis (Kufareva et al 2015, Imuno cell Biol, 93(4) 372). However, chemokines made to be oligomerization deficient by a single amino acid substitution, maintain wild type levels of chemotaxic stimulation. For example, many CC chemokines possess a proline at position 8 which when substituted to alanine abrogates dimerization without loss of chemotatic activity (Paavola et al 1998, JBC, V273; 33157). To disrupt the potential of PARK prochemokines to form dimers and facilitate their manufacture, PARK prochemokines is selected for those that possess a stable and active monomeric form or made dimerization deficient. This would facilitate the manufacture of a prochemokine tandem array format. Alternatively, a chemokine is manufactured as a homodimer using a linker from the C-terminus of the first monomer to the N-terminus of the second monomer similar to that used in scFv.

Tumors demonstrate tumor associated protease activity. Several tumors have been shown to possess tumor associated protease activity (Sudhan 2015, Choi 2012)(Table 1). tumor associated proteases include Cathepsins, plasminogen activators, plasmin, matrix metalloproteases and Kallikreins (Choi 2012). Some tumor associated proteases are described as prognostic factors. For example, elevated serum levels of Cathepsin L have been reported in patients with lung, pancreatic, and ovarian cancer when compared to healthy donors (Chenetal., 2011; Letoetal., 1997; Nishida et al., 1995; Siewinski et al., 2004; Tumminello et al., 1996; Zhangetal., 2011). Tumor associated proteases may be involved in metastasis. Tumor associated protease activity is the basis for the design of certain cancer therapeutics (Choi 2012). In addition, the expression of Cathepsin E is particularly high in stomach and pancreatic cancers, which is relevant herein. Propeptide cleavage by one or more tumor associated proteases is maximal proximal to the tumor site. Tumor associated proteases demonstrate increased propeptide cleavage at tumor sites. For example, a fluorescent probe that requires propeptide cleavage for fluorescence, was injected into a mouse tumor model (Hisiao 2006). Bioluminence peaked at tumor sites (a colon adenocarcinoma; HT-29 and a fibrosarcoma; HT-1080) indicating that the probe was predominantly cleaved at tumor sites by tumor associated protease activity.

When used for treat tumors, PARK is cleaved resulting in the release of active chemokines with maximal concentration localized at tumor sites. In another embodiment, a PARK comprises prochemokines released by active proteases associated with kidney, liver, lung or cardiac fibrosis, or Alzheimer's Disease. Such localized release may result in effective treatment with bioactive chemokines.

In on embodiment, a PARK disclosed herein may be encoded in a CAR vector and expressed in CAR-T or CAR-NK cells. In one embodiment, a PARK may be produced in mammalian cells which, if necessary, has a disrupted protease gene when it expresses the specific protease that cleaves a PARK.

### Example 3. PARK-Containing Therapeutic Protein

As depicted in FIG. 1 and Table 1, novel prochemokines is generated using recombinant DNA technology by fusing a propeptide to the N-terminus of chemokines. Chemokines converted to prochemokines may be CC, CXC, CX3C or C family chemokines, including but not limited to CCL1, CCL3, CCL5, CCL7, CCL14, CCL16, CCL19, CCL20, CCL21, CXCL8, CXCL9, CXCL10, CXCL12, CXCL16, XCL1, CX3CL1 and PROK2. Various propetides may be used to restrict cleavage to a specific tumor-associated protease(s) (Tables 1). A PARK may contain more than one type of propeptide that requires more than one tumor-associated protease to cleave and release one or more functional chemokines. For example, different protease cleavage sites may be positioned at the N- and C-terminal ends of the prochemokine. This may enable greater restriction of chemokine activation to tumor sites.

### Example 4. Assays for Identifying Propeptides Specifically Cleaved by Tumor Associated Proteases

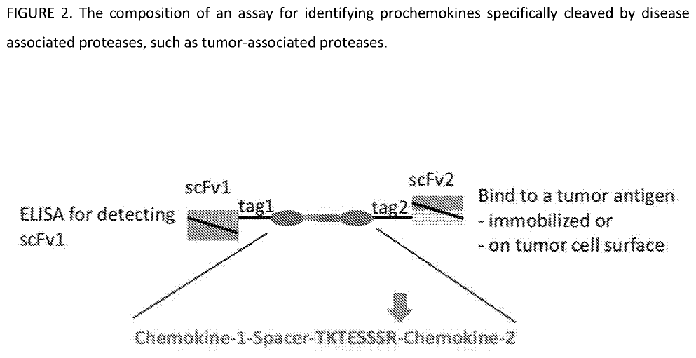

Propeptides may be selected from propeptide databases (e.g. http://www.cbs.dtu.dk/databases/propeptides/), synthesized from known substrate cleavage sequences, synthesized based on consensus protease cleavage sites or identified in a screen of peptides selectively cleaved by tumor-associated proteases. A screening assay to identify propetides specifically cleaved by tumor associated proteases may use a PARK that contains a reporter scFv with an epitope tag (tag1), such as flag, HA, and His. As shown in FIG. 2, this PARK may contain two chemokines linked by a spacer and one of several variations of a protease substrate site. The PARK is allowed to bind to a tumor antigen, either immobilized recombinant or expressed on the tumor cell surface. The tumor protease or a purified protease is tested for its ability to cleave the PARK over time by assaying the release of the reporter scFv as determined by ELISA. In the ELISA, cleavage is demonstrated by the binding of the released reporter scFv to immobilized antigen and detection with e.g. an anti-tag antibody coupled to HRP. The presence of the tumor binding moiety, either in the ELISA or on the tumor surface (by flow cytofluorimetry), is determined by use of an antibody specific for tag2. A library of substrate sequences may be screened for protease cleavage by using phage display of PARKs and sequencing DNA from the variants as described in Ratnikov et al., 2009. While the cleavage site may be fixed as shown in FIG. 2, this assay may be modified for demonstrating cleavage of PARK by proteases present in fibrosis or Alzheimer's disease. For example, by binding of the PARK reporter substrate to FAPα or Aβ. This assay may be used to generate amino acid substituted protease substrate sites to modify the rate of cleavage.

### Example 5. Configuration of PARK-Containing Proteins

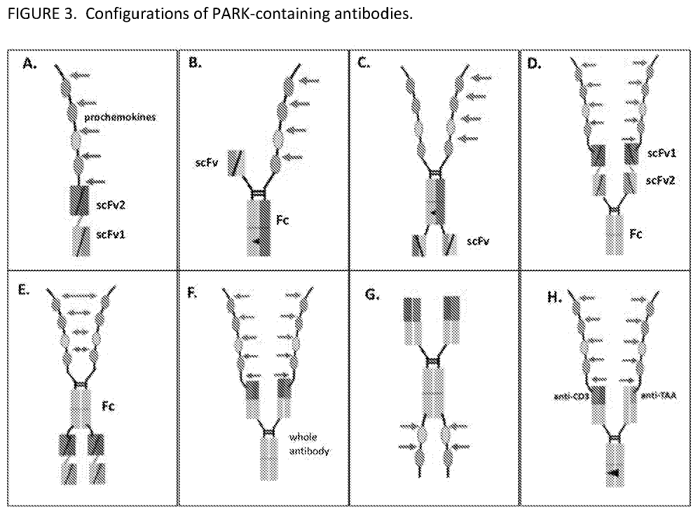

A PARK expression vector is constructed by having the sequences encoding the leader, one or more prochemokines, and a tumor, fibrosis or AD targeting protein in a single open reading frame for a single peptide. To possess additional binding activity that may increase therapeutic effect, such as in immune therapy involving antibodies, PARK may be added onto a therapeutic antibody. As examples, FIG. 3 displays several configurations of PARK-containing antibody proteins (i.e. PARK protein).

While the overall design of PARK proteins may vary, a PARK protein may possess a single prochemokine or several prochemokines in tandem as shown in FIG. 3A. Short spacer sequences, such as two or more glycine, may be inserted between tandem prochemokines. The mucin region of fractalkine may be incorporated at the C-terminus of PARK prochemokines.

A PARK protein may be a complex of two peptides, such as an antibody structure with one heavy chain and one light chain as shown in FIG. 3F-3H. A PARK construct may also encode an immunoglobulin Fc. The Fc may include two immunoglobulin CH2-CH3 domains that selectively associate with the other, such as in a knob-in hole-antibody, to yield bi-functional arms, as shown in FIG. 3B-3E. The sequences for three different knob-in-hole PARKs are listed (SEQ ID NO: 241, 251, 261, and 271). For example, one CH2-CH3 is to a moiety targeting a disease-associated antigen, such as tumor-associated antigen (TAA) and the second CH2-CH3 with prochemokines as shown in FIG. 3B. A PARK protein Fc domain may be of different isotypes and hence support ADCC or complement fixation.

A PARK protein may possess one or more prochemokines and one or more targeting antibodies, antibody fragments, or ligands. For example, FIG. 3A shows PARK prochemokines is presented in a tandem array tethered to one or more scFv to target one or more tumor or tumor stromal antigens. A PARK may have two different Ig CH2-CH3 domains that selectively bind with each other (knobs-in-hole Fc). As shown in FIG. 3B, one CH2-CH3 may be fused to a svFv fragment at its N-terminus for targeting the PARK to a disease-associated antigen, such as a tumor cell or stroma associated antigen. The other CH2-CH3 may possess one or more prochemokines fused to its N-terminus. The configuration in FIG. 3C shows a relatively symmetric structure of a PARK protein, of which each of two different Ig CH2-CH3 is linked to multiple prochemokine domains at its N-terminus and one scFv domain at its C-terminus and the two scFv domains may possess two different binding specificities.

Other variations may possess one or more prochemokines and tumor targeting moieties at the N- or C-terminus. FIGS. 3D-3E show that a PARK may possess an immunoglobulin Fc with one or more prochemokines or tumor targeting moieties at the N- or C-terminus. For example, the targeting moiety in FIG. 3E may be one or more native ligands, such as PD-1 or TIM3 that inhibit immune check points. FIGS. 3F-3H show that a PARK may possess a whole antibody with one or more prochemokines at the N- or C-terminus. PARKS may be bispecific and bind to two tumor antigens. Arrows indicate tumor protease cleavage sites. In one embodiment, a prochemokine is flanked by two different protease cleavage sites to increase specific on-tumor release of an active chemokine. In certain cases, protease cleavage sites may vary within a PARK such that the more C-terminal prochemokine cleavage sites are modified by amino acid substitution for decreased rate of cleavage to facilitate generating a chemokine gradient.

### Example 6. Constructing PARK-Expressing Vectors

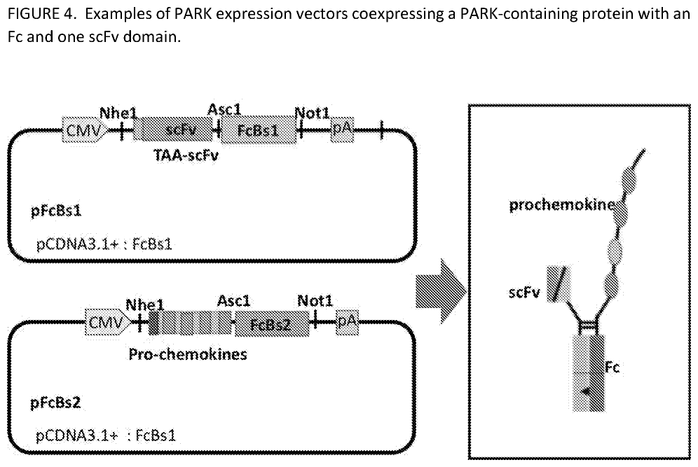

Expression constructs may be designed to secrete a PARK from mammalian cells as shown in FIG. 4. The mammalian cells harboring a disrupted protease gene may be advantageous for expressing an intact PARK or PARK protein. A protease gene, such as those identified in Table 1, may be disrupted by one of several methods, such as CRISPR. A PARK may also be produced from insect cells or yeast that do not express a protease and yet cleaves the engineered PARK protease sites. PARK constructs may express one or more prochemokines and ligands targeting one or more tumor, fibrosis or AD associated antigens. TAA-scFv is a tumor associated antigen specific scFv.

### Example 7. Modifying the Protease Cleavage Site

A PARK may possess a prochemokine with a modified protease cleavage site sequence as shown in FIG. 3 at different locations as indicated by arrows. The modified sites may decrease the rate of cleavage and enable additional time for PARK localization to a tumor before releasing active chemokines. The modified sites may occur in the prochemokines proximal to the targeting moiety such that these chemokines would be the last to be released allowing for a more sequential release and gradient. Non-optimal protease substrates for uPA and tPA have been reported (Lui et al 2001, JBC 276 17976).

### Example 8. Examples of PARK Proteins for Treating Diseases, Including Cancer, Fibrosis, and Alzheimer's Disease

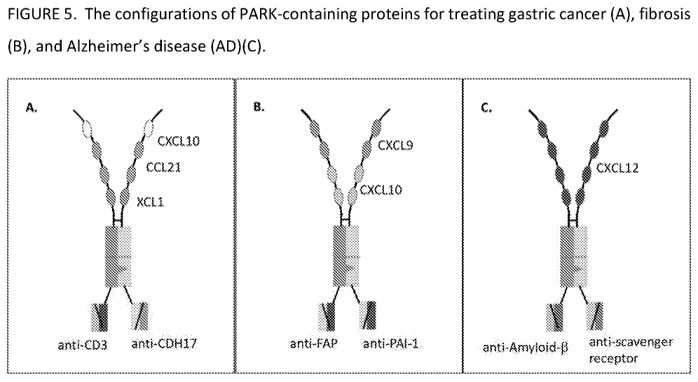

It is now generally accepted that an anti-CD3 moiety redirects T cell killing to the tumor target or cancer associate fibroblasts (CAF) and that anti-CD11b may support phagocytosis of tumor cells. Similarly, an anti-PAI-1 moiety may increase activity of uPA and plasmin for increased release of PARK chemokines and degradation of fibrotic extracellular matrix, and an anti-scavenger receptor may stimulate uptake of Aβ. To configure these functionalities into a PARK protein, the structure in FIG. 3C is selected for producing PARK therapeutic proteins. As shown in FIG. 5A-5C, all three PARK proteins comprise prochemokines in tandem at the N-terminus to knobs-in-hole Fc and two scFv binding domains at the C-terminus to the Fc. For treating gastric cancer, the two scFv domains possess binding specificities to CD3 of T cells and CDH17 of gastric cancer cells, while the prochemokines possess the mature activities of CXCL10, CCL21, and XCL1 (FIG. 5A, SEQ ID NO: 221-222). For treating fibrosis, the two scFv domains possess binding specificities to FAP and PAI-1 of tissue fibroblasts, while the prochemokines possess the mature activities of CXCL9 and CXCL10 (FIG. 5B, SEQ ID NO: 231-232). For treating AD, the two scFv domains possess binding specificities to Amyloid-β and scavenger receptor, while the prochemokines possess the mature activities of CXCL12 (FIG. 5C).

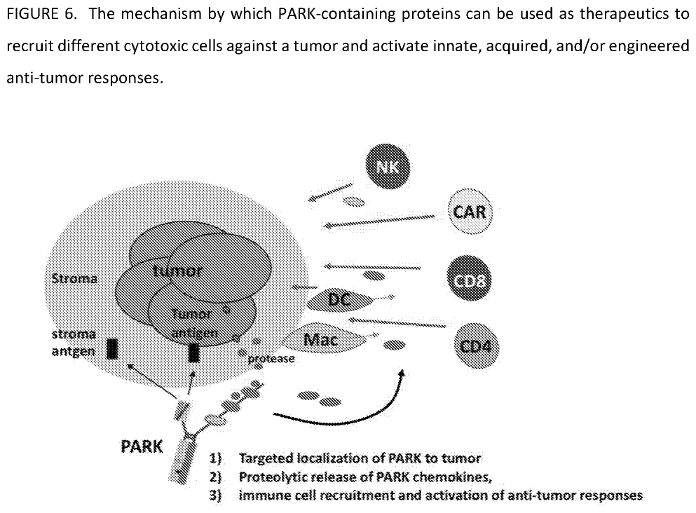

In each configuration as described either in FIG. 3C or FIG. 5, the targeting moiety may be an scFv, nanobody or endogenous ligand domain that binds a lesion specific antigen as shown in FIG. 6. Example chemokines are those that are capable of recruiting cells that will support an anti-tumor response but not cell types that may dampen an anti-tumor response, such as Tregs. Example chemokines include those that demonstrate anti-tumor activity, especially regression when injected into tumors (Homey, 2002). PARK constructs that target tumor antigens may also be conjugated to cytotoxic drugs to enhance anti-tumor activity.

Codons encoding a PARK may be optimized for expression and production in human, bacterial or yeast cells using appropriate expression vectors. PARK proteins may possess a protein tag, for example, an amino acid sequence comprising from about 8 to about 10 histidines, streptag-2 or immunoglobulin Fc, which may be utilized for their purification by standard techniques.

An effective concentration of purified PARK proteins may be administered to cancer, fibrosis or Alzheimer's disease (AD) patients by different routes of administration including intravenous, intraperitoneal, subcutaneous, intracerebroventricular, intravitreal, intrathecal or intratumorr. PARK proteins may be administered in a single or in multiple doses. PARK may be administered over a concentration range. PARK may be used as a mono-therapeutic or in combination with other immunotherapies, cell therapies, chemotherapies or drug therapies. For example, PARK may be combined with CAR T or CAR NK cells to enable greater tumor localization.

### Example 9. PARK as a Platform Technology for Cancer Immune Therapy

The in vitro release of PARK chemokines will be demonstrated following PARK binding to a tumor antigen coated microtiter well and the addition of a tumor-associated protease. In addition, chemokine release will be demonstrated following PARK binding to a tumor cell that expresses a specific protease.

The initial PARK constructs may contain prochemokines that are substrates for uPA, a cathepsin and/or an MMP. Chemokine release may be measured over time by a chemokine ELISA and by a cell-based calcium mobilization or chemotaxis assay. Negative controls will include no protease and a PARK without protease cleavage sites. Release of the Fc portion of PARK will also be determined by ELISA to demonstrate that PARK binding to the tumor antigen is not altered by the protease.

As of the in vivo example, PARK will be administered into a mouse xenograft cancer model that typically exhibits low levels of lymphocyte infiltration and in which tumor cells or tumor stroma have been determined to express the PARK specific tumor antigen and the protease specific for the chemokine propetide.

Increased tumor lymphocyte infiltration is determined over time by immunohistochemistry (IHC) or by bioluminescence using a mouse model with fluorescently tagged lymphoid cells. Alternatively, human lymphoid cells are injected iv and tumor infiltration measure by IHC with human specific anti-lymphoid antibodies or bioluminescence if cells are appropriately tagged. In the latter case PARK may contain human chemokines.

PARK may be used to treat any cancer type to induce or enhance an anti-tumor immune response. PARK may preferably be used to treat solid tumors that demonstrate low levels of lymphocyte infiltrate. PARK may be used to treat lung, colon, liver, gastric, pancreatic, prostate, breast, ovarian, brain or other cancer types.

Many tumor types do not develop a sufficient anti-tumor immune response, and some demonstrate low levels of lymphocyte infiltrate. PARK benefits cancer patients by recruiting and/or activating one or more cell types capable of anti-tumor activity such as dendritic cells, T cells, NK cells, and macrophages to a tumor as shown in FIG. 6. PARK treatment could dampen tumor mediated immunosuppression and facilitate a more effective anti-tumor immune response. PARK can be used to enable greater homing of CART or CAR NK cells to tumors. This will increase the efficiency of CAR cells by concentrating them in a tumor, especially in tumors that do not attract lymphoid cells or other immune cell types. This may allow for a reduction in the number of CAR cells that need to be administered to achieve a complete and durable anti-tumor response. The greater tumor localization of CAR cells and the reduction of CAR cells necessary for a response may enable decreased CAR mediated toxicity to normal tissue.

Alternatively, prochemokines are linked to a bispecific antibody targeting a tumor associated antigen and T or NK cell receptor. This enables an additional PARK function; in addition to localizing immune cells to the tumor site, the anti-CD3 will stimulate T cell mediated tumor killing. Anti-CD3 may be replaced with an anti-NK cell receptor to stimulate NK mediated tumor killing. Conversely, a bispecific PARK prochemokine may sterically hinder binding to T or NK cells. The prochemokines may be fused to the TCR or NK cell targeting moieties such that their binding is blocked due to prochemokine steric hindrance. The bispecific antibody is localized to a tumor via the target moiety. At the tumor site the prochemokines are cleaved by a tumor associated protease allowing the anti-TCR or anti-NK receptor to bind and activate tumor localized killing. This may enable low levels of undesirable off-tumor toxicity.

### Example 10. PARK as a Flexible Platform

PARK is a flexible platform that can use various chemokines capable of recruiting different cell types of the innate and acquired immune systems (Table 2). One or more chemokines may be incorporated into a PARK protein to maximally enhance an anti-tumor response. For example, chemokines may be selected that recruit both antigen presenting and tumor cytotoxic cells but not Tregs which can dampen an anti-tumor immune response. A PARK may recruit both cytotoxic T cells and NK cells.

PARK proteins can be designed to target a broad range of antigens to target different tumors, fibrosis or AD (Table 3). A single PARK may contain specificity for one or more tumor associated antigens by possessing two scFv with different specificities as shown in FIG. 3. A PARK may also enhance an anti-tumor immune response by blocking the interaction of immune check point inhibitors. For example, PARK scFv may bind PD-L1 and/or PD-L2 (Table 3).

PARK proteins may inhibit tumor growth or possess tumor cytotoxicity activity. In this situation, the PARK targeting moiety may block the activity of a growth factor receptor, e.g. EGF or VEGF receptors, stimulate an apoptotic response, e.g. Fas ligand or mediate cytotoxicity by ADCC, complement fixation or as a drug conjugate. As a drug conjugate PARK may be conjugated to irinotecan, auristatins, PBDs, maytansines, amantins, spliceosome inhibitors and other chemotherapeutic agents.

PARK proteins may possess different propeptides to restrict cleavage to specific proteases associated with certain tumor types. Thus, PARK activity may be tailored for different cancer types. A PARK may contain more than one type of propeptide that require more than one tumor associated protease to cleave and release a functional chemokine. This may enable greater restriction of chemokine activation to tumor sites.

Several cancer treatments effect lymphocyte infiltration into tumors including antibodies specific for immune checkpoint inhibitors, anti-angiogenetic agents, treatments that deplete Treg cells and oncolytic virotherapy (Oelkrug 2014). None of these treatments directly control the type and concentrations of specific chemokines in a tumor site. Some of these treatments are not anticipated to be effective to a tumor with low levels of lymphoid cells prior to treatment.

### REFERENCES

1. Jung I K, Kim S S, Suh D S, Kim K H, Lee C H, Yoon M S. 2014. Tumor-infiltration of T-lymphocytes is inversely correlated with clinicopathologic factors in endometrial adenocarcinoma. Obstet Gynecol Sci. July; 57(4):266-73.
2. Homey B, Anja Müller & Albert Zlotnik. 2002 Chemokines: agents for the immunotherapy of cancer? Nature Reviews Immunology 2, 175-184
3. Sudhan D R, S iemann DW. 2015 Cathepsin L targeting in cancer treatment.
4. Pharmacol Ther. November; 155:105-16.
5. Gong Y, Chippada-Venkata U D, Oh W K. 2014 Roles of matrix metalloproteinases and their natural inhibitors in prostate cancer progression Cancers (Basel). June 27; 6(3):1298-327.
6. Hsiao J K, Law B, Weissleder R, Tung C H. 2006. In-vivo imaging of tumor associated urokinase-type plasminogen activator activity. J Biomed Opt. May-June; 11(3):34013.
7. Yixuan Gong, Uma D. Chippada-Venkata and William K. Oh 2014 Roles of Matrix Metalloproteinases and Their Natural Inhibitors in Prostate Cancer Progression, Cancers, 6, 1298-1327
8. Tauro M, McGuire J, Lynch C C. 2014 New approaches to selectively target cancer-associated matrix metalloproteinase activity. Cancer Metastasis Rev. December; 33(4):1043-57.
9. Góra J, Latajka R. 2015. Involvement of cysteine proteases in cancer Curr Med Chem.; 22(8):944-57.
10. The roles of ADAMTS in angiogenesis and cancer. Tumour Biol. 2015 June; 36(6):4039-51. doi: 10.1007/s13277-015-3461-8. Epub 2015 Apr. 28.
11. Peng D, Kryczek I, Nagarsheth N, Zhao L, Wei S, Wang W, Sun Y, Zhao E, Vatan L, Szeliga W, Kotarski J, Tarkowski R, Dou Y, Cho K, Hensley-Alford S, Munkarah A, Liu R, Zou W. 2015, Epigenetic silencing of TH1-type chemokines shapes tumour immunity and immunotherapy. Nature. November 12; 527(7577):249-53
12. Trusevych E H, MacNaughton W K 2015 Proteases and their receptors as mediators of inflammation-associated colon cancer. Curr Pharm Des.; 21(21):2983-92.
13. Ueki N, Lee S, Sampson N S, Hayman M J. 2013 Selective cancer targeting with prodrugs activated by histone deacetylases and a tumour-associated protease. Nat Commun.; 4:2735.
14. Sudhan D R, Siemann D W, Cathepsin L targeting in cancer treatment. Pharmacol Ther. 2015 November; 155:105-16. doi: 10.1016/j.pharmthera.2015.08.007. Epub 2015 Aug. 20.
15. Kushlinskii N E, Gershtein E S, Korotkova E A, Prorokov V V., Prognostic role of tumor-associated proteases in colorectal cancer. Bull Exp Biol Med. 2013 January; 154(3):365-9.
16. Gershtein E S, Korotkova E A, Prorokov V V, Kushlinskii N E, Tumor associated proteases—prognostic markers of colorectal cancer. Klin Lab Diagn. 2013 October; (10):43-7, 5-10
17. Allgayer H, Babic R, Grutzner K U, Beyer B C, Tarabichi A, Schildberg F W, Heiss M M, Tumor-associated proteases and inhibitors in gastric cancer: analysis of prognostic impact and individual risk protease patterns. Clin Exp Metastasis. 1998 January; 16(1):62-73.
18. Vakili J, Standker L, Detheux M, Vassart G, Forssmann W G, Parmentier M 2001 Urokinase plasminogen activator and plasmin efficiently convert hemofiltrate CC chemokine 1 into its active. J Immunol. September 15; 167(6):3406-13.
19. Blain K Y, Kwiatkowski W, Zhao Q, La Fleur D, Naik C, Chun T W, Tsareva T, Kanakaraj P, Laird M W, Shah R, George L, Sanyal I, Moore P A, Demeler B, Choe S, Biochemistry. 2007 Sep. 4; 46(35):10008-15. Epub 2007 Aug. 11; Structural and functional characterization of CC chemokine CCL14.
20. Detheux M, Standker L, Vakili J, Munch J, Forssmann U, Adermann K, Pohlmann S, Vassart G, Kirchhoff F, Parmentier M, Forssmann W G 2000 Natural proteolytic processing of hemofiltrate CC chemokine 1 generates a potent CC chemokine receptor (CCR)1 and CCR5 agonist with anti-HIV properties. J Exp Med. November 20; 192(10):1501-8.

TABLE 1
Examples of various proteases that may cleave and activate PARK prochemokines

| Protease | Tumor expression | Localization | Substrates |
|---|---|---|---|
| uPA/plasmin | Cervical, colorectal, | Secreted and | TKTESSSR |
| | gastric, prostate | membrane (uPAR) | GGSGRSANAKC. . . |
| Cathepsin-L, -E, -K, -B, | Many types of cancer | Vesicles, secreted, | XXX aromatic FR XXX |
| and -V | | pericellular in cancer | XXX aromatic RR XXX. . . |
| MMP-1, -8, and -13 | Breast | Pericellular | GPQGIWGQ. . . |
| MMP-2, and -9 | Breast, colorectal, | Pericellular | L/I/HXXhydrophobic |
| | lung, | | PQGIAGQ |
| | malignant gliomas, | | PLGVRGK |
| | ovarian | | SVVAPPPVVLL |
| | | | PLGL. . . |
| | | | GPLG-VRGK |
| | | | HPVG-LLAR |
| MMP-14, -15, 16, -17, | Many forms of cancer | Membrane | PQD-LL |
| and -24 | | | QG-LLD |
| | | | VS-MSS. . . |
| Kallikreins; PSA (hk3), | Many forms of cancer | Secreted | SSKLQ |
| hK10, hK15 | | | |
| Fibroblast activation | Many forms of cancer | Tumor-associated | TAGPNQ |
| protein (FAP) | | fibroblast cell | TSGPSQ. . . |
| | | membranes | |

Modified from Choi et al 2012: X=any amino acid; *Substrate sequences may be selected from protease databases, such as PMAP (http://pmap.burnham.org/proteases)

TABLE 2
Chemokines, target cells and functions.

| 1. C-C Chemokine | Receptor | Distribution | Functions |
|---|---|---|---|
| CCL3, CCL5, CCL7, | CCR1 | monocytes, memory | T cell and monocyte migration; |
| CCL8, CCL13-16 | | T cells, Th1, NK | hypersenstivity; innate and adaptive |
| | | | immunity; inflammation |
| CCL2, CCL7, CCL8, | CCR2 | monocytes, memory | T cell and monocyte migration; innate and |
| CCL13 | | T cells, basophils, | adaptive immunity; Th1 inflammation |
| | | pDC | |
| CCL5, CCL7; | CCR3 | eosinophils, | eosinophil and basophil migration; allergic |
| CCL11, CCL15-16, | | basophils | inflammation; Th2 response |
| CCL24, CCL26 | | | |
| CCL17, CCL22 | CCR4 | Th2 cells, | T cell and monocyte migration; allergic |
| | | T_{reg} eosinophils, | inflammation; T_{reg} retention; skin homing; |
| | | basophils, DC, T_{reg} | expressed on CD4 Th2 cells |
| CCL3; CCL4; CCL5; | CCR5 | monocytes, Th1 | Th1 response, adaptive immunity; |
| CCL8 | | cells, NK | inflammation, HIV infection |
| CCL20 | CCR6 | memory T cells, B | DC migration, memory T cells, Th17 cells at |
| | | cells, Thl7, | site of inflammation |
| | | immature mDC | |
| CCL19. CCL21 | CCR7 | naive T, B, mature | T cell and DCs homing to secondary |
| | | mDC, Thl, Th2, T_{reg} | lymphoid tissue; lymphoid development |
| CCL1 | CCR8 | monocytes, Th2, | T cell trafficking; Th2 response |
| | | T_{reg}, NK | |
| CCL25 | CCR9 | DC, memory T cells, | T cell homing to gut and thymus tolerogenic |
| | | thymocytes | DCs |
| CCL27, CCL28 | CCR10 | memory T cells, T_{reg} | T cell homing to skin and bowel |
| 2. CXC chemokine | CXC | | |
| CXCL6, CXCL7, | CXCR1 | PMN, monocytes, | neutrophil migration; innate immunity; |
| CXCL8 | | mast cells | acute inflammation |
| CXCLl-3; CXCL5-8 | CXCR2 | PMN, monocytes, | neutrophil migration; innate immunity; |
| | | mast cells | acute inflammation; angiogenesis |
| CXCL9, CXCL10, | CXCR3 | memory T cells, Th1, | T cell recruitment; adaptive immunity; Th1, |
| CXCL11 | | Th2, Th17, T_{reg}, NKT | Th2, Th17, T_{reg} inflammation |
| CXCL12 | CXCR4 | T and B cells, | stem cell migration; B cell lymphopoiesis |
| | | monocytes, stem | |
| | | cells, NKT | |
| CXCL13 | CXCR5 | B cells | B cell homing in lymphoid organ |
| CXCL16 | CXCR6 | memory T cells, Th1, | T cell migration |
| | | NK, NKT | |
| | CXCR7 | | |
| 3. CX3C, XC | CX3C, XC | | |
| chemokine | Receptor | | |
| CX3CL1 | CX3CR1 | monocytes, Th1, NK | T cell and NK cell trafficking and adhesion; |
| | | | innate and adaptive immunity; Thl |
| | | | inflammation |
| XCL1-2 | XCR1 | NK | NK cell recruitment |

For references, see Oo 2012.

TABLE 3
Examples of PARK targeting specificities or ligands

| Specificity of scFv or other | Native ligands |
|---|---|
| antibody fragments in PARKs | in PARKs |
| PD-L1 | PD-1 |
| PD-L2 | TIM3 |
| CDH17 | EGF1, EGF2 |
| TROP-2 | VEGF |
| Growth factor receptors e.g. | IGF |
| EGFR, HER2, VEGFR, IGF1R | |
| B7H3 | Cytokines |
| EpCAM | c-METErb3 |
| PSMA | Tigit |
| Glypican-3 | Chemokines |
| CEAs e.g. CEACAM5 | Cytokines |
| Gangliosides e.g. GD2, GM3, GM2 | |
| CD19 + CD22 | |
| Fibroblast activation protein-1 (FAP) | |
| Amyloid beta | |
| Scavenger receptors e.g. | |
| SR-A1, SR-B1, SR-F, SR-L | |
| Phagocytic receptors, e.g. | |
| CD11b/CD18, IgFc receptors | |
| Integrins e.g. αVβ3, α6β4 | |

SEQUENCE LISTING

SEQ ID NO: 1
NATIVE CCL14 IS A PRO-CCL14 COMPRISING A CCL14 LEADER (SEQ ID NO: 2), A CCL14 PROPEPTIDE
(SEQ ID NO: 3), AND A BIOACTIVE CCL14 CHEMOKINE (SEQ ID NO: 4) SEQUENCES
MKISVAAIPFFLLITIALGTKTESSSRGPYHPSECCFTYTTYKIPRQRIMDYYETNSQC
SEQ ID NO: 2
CCL14 LEADER SEQUENCE
MKISVAAIPFFLLITIALG
SEQ ID NO: 3
CCL14 PROPEPTIDE SEQUENCE
TKTESSSR
SEQ ID NO: 4
CCL14 MATURE ACTIVE CHEMOKINE SEQUENCE
MKISVAAIPFFLLITIALGTKTESSSRGPYHPSECCFTYTTYKIPRQRIMDYYETNSQC
SEQ ID NO: 5
NATIVE RANTES (also known as CCL5) COMPRISE A CCL5 LEADER (SEQ ID NO: 6) AND A BIOACTIVE
CCL5 CHEMOKINE (SEQ ID NO: 7) SEQUENCES
MKVSAAALAVILIATALCAPASASPYSSDTTPCCFAYIARPLPRAHIKEYFYTSGKCSNPAVVFVTRKNRQVCANPEKKWV
REYINSLEMS
SEQ ID NO: 6
A CCL5 LEADER SEQUENCE
MKVSAAALAVILIATALCAPASA
SEQ ID NO: 7
A BIOACTIVE RANTES/CCL5 CHEMOKINE SEQUENCE
SPYSSDTTPCCFAYIARPLPRAHIKEYFYTSGKCSNPAVVFVTRKNRQVCANPEKKWVREYINSLEMS
SEQ ID NO: 8
A PRO-RANTES (PRO-CCL5) IS A RECOMBINANT PROTEIN COMPRISING A CCL14 LEADER (SEQ ID NO: 2),
A CCL14 PROPEPTIDE (SEQ ID NO: 3), AND A BIOACTIVE RANTES/CCL5 CHEMOKINE (SEQ ID NO: 7)
SEQUENCES
MKISVAAIPFFLLITIALGTKTESSSRSPYSSDTTPCCFAYIARPLPRAHIKEYFYTSGKCSNPAVVFVTRKNR
QVCANPEKKWVREYINSLEMS
SEQ ID NO: 11
A BIOACTIVE CCL1 CHEMOKINE SEQUENCE
KSMQVPFSRCCFSFAEQEIPLRAILCYRNTSSICSNEGLIFKLKRGKEACALDTVGWVQRHRKMLRHCPSKRK
SEQ ID NO: 12
A BIOACTIVE CCL2 CHEMOKINE SEQUENCE
QPDAINAPVTCCYNFTNRKISVQRLASYRRITSSKCPKEAVIFKTIVAKEICADPKQKWVQDSMDHLDKQTQTPKT
SEQ ID NO: 13
A BIOACTIVE CCL3 CHEMOKINE SEQUENCE
SLAADTPTACCFSYTSRQIPQNFIADYFETSSQCSKPGVIFLTKRSRQVCADPSEEWVQKYVSDLELSA
SEQ ID NO: 14
A BIOACTIVE CCL20 CHEMOKINE SEQUENCE
ASNFDCCLGYTDRILHPKFIVGFTRQLANEGCDINAIIFHTKKKLSVCANPKQTWVKYIVRLLSKKVKNM
SEQ ID NO: 15
A BIOACTIVE CCL21 CHEMOKINE SEQUENCE
SDGGAQDCCLKYSQRKIPAKVVRSYRKQEPSLGCSIPAILFLPRKRSQAELCADPKELWVQQLMQHLDKTPSPQKPAQG
CRKDRGASKTGKKGKGSKGCKRTERSQTPKGP
SEQ ID NO: 16
A BIOACTIVE CXCL9 CHEMOKINE SEQUENCE
TPVVRKGRCSCISTNQGTIHLQSLKDLKQFAPSPSCEKIEIIATLKNGVQTCLNPDSADVKELIKKWEKQVSQKKKQKNGK
KHQKKKVLKVRKSQRSRQKKTT
SEQ ID NO: 17
A BIOACTIVE CXCL10 CHEMOKINE SEQUENCE
VPLSRTVRCTCISISNQPVNPRSLEKLEIIPASQFCPRVEIIATMKKKGEKRCLNPESKAIKNLLKAVSKERSKRSP
SEQ ID NO: 18
A BIOACTIVE CXCL16 CHEMOKINE SEQUENCE
SVTGSCYCGKRISSDSPPSVQFMNRLRKHLRAYHRCLYYTRFQLLSWSVCGGNKDPWVQELMSCLDLKECGHAYS
SEQ ID NO: 19
A BIOACTIVE XCL1 CHEMOKINE SEQUENCE
VGSEVSDKRTCVSLTTQRLPVSRIKTYTITEGSLRAVIFITKRGLKVCADPQATWVRDVVRSMDRKSNTRNNMIQTKPTG
TQQSTNTAVTLTG
SEQ ID NO: 20
A BIOACTIVE CX3CL1 CHEMOKINE SEQUENCE
QHHGVTKCNITCSKMTSKIPVALLIHYQQNQASCGKRAIILETRQHRLFCADPKEQWVKDAMQHLDRQAAALTRNG
SEQ ID NO: 111
A PRO-CCL1 IS A RECOMBINANT PROTEIN COMPRISING A CCL14 LEADER (SEQ ID NO: 2), A CCL14
PROPEPTIDE (SEQ ID NO: 3), AND A BIOACTIVE CCL1 CHEMOKINE (SEQ ID NO: 11) SEQUENCES
MKISVAAIPFFLLITIALGTKTESSSRKSMQVPFSRCCFSFAEQEIPLRAILCYRNTSSICSNEGLIFKLKRGK
EACALDTVGWVQRHRKMLRHCPSKRK
SEQ ID NO: 121
A PRO-CCL2 IS A RECOMBINANT PROTEIN COMPRISING A CCL14 LEADER (SEQ ID NO: 2), A CCL14
PROPEPTIDE (SEQ ID NO: 3), AND A BIOACTIVE CCL2 CHEMOKINE (SEQ ID NO: 12) SEQUENCES
MKISVAAIPFFLLITIALGTKTESSSRQPDAINAPVTCCYNFTNRKISVQRLASYRRITSSKCPKEAVIFKTIV
AKEICADPKQKWVQDSMDHLDKQTQTPKT
SEQ ID NO: 131
A PRO-CCL3 IS A RECOMBINANT PROTEIN COMPRISING A CCL14 LEADER (SEQ ID NO: 2), A CCL14
PROPEPTIDE (SEQ ID NO: 3), AND A BIOACTIVE CCL3 CHEMOKINE (SEQ ID NO: 13) SEQUENCES
MKISVAAIPFFLLITIALGTKTESSSRSLAADTPTACCFSYTSRQIPQNFIADYFETSSQCSKPGVIFLTKRSR
QVCADPSEEWVQKYVSDLELSA
SEQ ID NO: 141
A PRO-CCL20 IS A RECOMBINANT PROTEIN COMPRISING A CCL14 LEADER (SEQ ID NO: 2), A CCL14
PROPEPTIDE (SEQ ID NO: 3), AND A BIOACTIVE CCL20 CHEMOKINE (SEQ ID NO: 14) SEQUENCES
MKISVAAIPFFLLITIALGTKTESSSRASNFDCCLGYTDRILHPKFIVGFTRQLANEGCDINAIIFHTKKK
LSVCANPKQTWVKYIVRLLSKKVKNM
SEQ ID NO: 151
A PRO-CCL21 IS A RECOMBINANT PROTEIN COMPRISING A CCL14 LEADER (SEQ ID NO: 2), A CCL14
PROPEPTIDE (SEQ ID NO: 3), AND A BIOACTIVE CCL21 CHEMOKINE (SEQ ID NO: 15) SEQUENCES
MKISVAAIPFFLLITIALGTKTESSSRSDGGAQDCCLKYSQRKIPAKVVRSYRKQEPSLGCSIPAILFLPRKRS
QAELCADPKELWVQQLMQHLDKTPSPQKPAQGCRKDRGASKTGKKGKGSKGCKRTERSQTPKGP
SEQ ID NO: 161
A PRO-CXCL9 IS A RECOMBINANT PROTEIN COMPRISING A CCL14 LEADER (SEQ ID NO: 2), A CCL14
PROPEPTIDE (SEQ ID NO: 3), AND A BIOACTIVE CXCL9 CHEMOKINE (SEQ ID NO: 16) SEQUENCES
MKISVAAIPFFLLITIALGTKTESSSRTPVVRKGRCSCISTNQGTIHLQSLKDLKQFAPSPSCEKIEIIATLKNGVQTCLNPDS
ADVKELIKKWEKQVSQKKKQKNGKKHQKKKVLKVRKSQRSRQKKTT
SEQ ID NO: 171
A PRO-CXCL10 IS A RECOMBINANT PROTEIN COMPRISING A CCL14 LEADER (SEQ ID NO: 2), A CCL14
PROPEPTIDE (SEQ ID NO: 3), AND A BIOACTIVE CXCL10 CHEMOKINE (SEQ ID NO: 17) SEQUENCES
MKISVAAIPFFLLITIALGTKTESSSRVPLSRTVRCTCISISNQPVNPRSLEKLEIIPASQFCPRVEIIATMKKKGEKRCLNPES
KAI KNLLKAVSKERSKRSP
SEQ ID NO: 181
A PRO-CXCL16 IS A RECOMBINANT PROTEIN COMPRISING A CCL14 LEADER (SEQ ID NO: 2), A CCL14
PROPEPTIDE (SEQ ID NO: 3), AND A BIOACTIVE CXCL16 CHEMOKINE (SEQ ID NO: 18) SEQUENCES
MKISVAAIPFFLLITIALGTKTESSSRSVTGSCYCGKRISSDSPPSVQFMNRLRKHLRAYHRCLYYTRFQLLSWSVCGGNKD
PWVQELMSCLDLKECGHAYS
SEQ ID NO: 191
A PRO-XCL1 IS A RECOMBINANT PROTEIN COMPRISING A CCL14 LEADER (SEQ ID NO: 2), A CCL14
PROPEPTIDE (SEQ ID NO: 3), AND A BIOACTIVE XCL1 CHEMOKINE (SEQ ID NO: 19) SEQUENCES
MKISVAAIPFFLLITIALGTKTESSSRVGSEVSDKRTCVSLTTQRLPVSRIKTYTITEGSLRAIFITKRGLKVCADPQATWVR
DVVRSMDRKSNTRNNMIQTKPTGTQQSTNTAVTLTG
SEQ ID NO: 201
A PRO-CX3CL1 IS A RECOMBINANT PROTEIN COMPRISING A CCL14 LEADER (SEQ ID NO: 2), A CCL14
PROPEPTIDE (SEQ ID NO: 3), AND A BIOACTIVE CX3CL1 CHEMOKINE (SEQ ID NO: 20) SEQUENCES
MKISVAAIPFFLLITIALGTKTESSSRQHHGVTKCNITCSKMTSKIPVALLIHYQQNQASCGKRAIILETQHRLFCADPKE
QWVKDAMQHLDRQAAALTRNG
SEQ ID NO: 211
PROCHEMOKINES IN TANDEM: THE SEQUENCE OF PRO-CCL21-XCL1
MKISVAAIPFFLLITIALGTKTESSSRGSDGGAQDCCLKYSQRKIPAKVVRSYRKQEPSLGCSIPAILFLPRKRSQAELCADPK
ELWVQQLMQHLDKTPSPQKPAQGCRKDRGASKTGKKGKGSKGCKRTERSQTPKGPGGGTKTESSSRGVGSEVSDKR
TCVSLTTQRLPVSRIKTYTITEGSLRAVIFITKRGLKVCADPQATWVRDVVRSMDRKSNTRNNMIQTKPTGTQQSTNTA
VTLTG
SEQ ID NO: 221
A PARK PROTEIN, CCL21-XCL1-FCKNOB-αCD3 AS SHOWN IN FIG. 5A, IS A RECOMBINANT PROTEIN
COMPRISING PRO-CXCL10-CCL21 AND AN ANTI-CD3 SCFV DOMAIN SEQUENCES.
MKISVAAIPFFLLITIALGTKTESSSRGVPLSRTVRCTCISISNQPVNPRSLEKLEIIPASQGCPRVEIIATMKKKGEKRCLNPE
SKAIKNLLKAVSKERSKRSPGGGTKTESSSRSDGGAQDCCLKYSQRKIPAKVVRSYRKQEPSLGCSIPAILFLPRKRSQAEL
CADPKELWVQQLMQHLDKTPSPQKPAQGCRKDRGASKTGKKGKGSKGCKRTERSQTPKGPGGGTKTESSSRSDGGA
QDCCLKYSQRKIPAKVVRSYRKQEPSLGCSIPAILFLPRKRSQAELCADPKELWVQQLMQHLDKTPSPQKPAQGCRKDR
GASKTGKKGKGSKGCKRTERSQTPKGPGGGTKTESSSRVGSEVSDKRTCVSLTTQRLPVSRIKTYTITEGSLRAVIFITKRG
LKVCADPQATWVRDVVRSMDRKSNTRNNMIQTKPTGTQQSTNTAVTLTGGAPGGEPKSSDKTHTCPPCPAPELLGG
PSVFLFPPKPKDTLMISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPCPEEMTKNQVSLWCLVKGFYPSDIAVEWESNGQPENNYKT
TPPVLDSDGSFFLYSRLTVDKSRWQEGNVFSCSVMHEALHNHYTQKSLSLSLGKPAGGGGSEVQLVESGGGLVQPGGS
LRLSCAASGYSFTGYTMNWVRQAPGKGLEWVALINPYKGVSTYNQKFKDRFTISVDKSKNTAYLQMNSLRAEDTAVYY
CARSGYYGDSDWYFDVWGQGTLVTVSSGGGGSGGGGSGGGGSDIQMTQSPSSLSASVGDRVTITCRASQDIRNYLN
WYQQKPGKAPKLLIYYTSRLESGVPSRFSGSGSGTDYTLTISSLQPEDFATYYCQQGNTLPWTFGQGTKVEIK*
SEQ ID NO: 222
A PARK PROTEIN, CCL21-XCL1-FCHOLE-αCDH17 AS SHOWN IN FIG. 5A, IS A RECOMBINANT
PROTEIN COMPRISING PRO-CXCL10-CCL21 AND AN ANTI-CDH17 SCFV DOMAIN SEQUENCES.
MKISVAAIPFFLLITIALGTKTESSSRGVPLSRTVRCTCISISNQPVNPRSLEKLEIIPASQFCPRVEIIATMKKKGEKRCLNPE
SKAIKNLLKAVSKERSKRSPGGGTKTESSSRSDGGAQDCCLKYSQRKIPAKVVRSYRKQEPSLGCSIPAILFLPRKRSQAEL
CADPKELWVQQLMQHLDKTPSPQKPAQGCRKDRGASKTGKKGKGSKGCKRTERSQTPKGPGGGTKTESSSRSDGGA
QDCCLKYSQRKIPAKVVRSYRKQEPSLGCSIPAILFLPRKRSQAELCADPKELWVQQLMQHLDKTPSPQKPAQGCRKDR
GASKTGKKGKGSKGCKRTERSQTPKGPGGGTKTESSSRVGSEVSDKRTCVSLTTQRLPVSRIKTYTITEGSLRAVIFITKRG
LKVCADPQATWVRDVVRSMDRKSNTRNNMIQTKPTGTQQSTNTAVTLTGGAPGGEPKSSDKTHTCPPCPAPELLGG
PSVFLFPPKPKDTLMISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVCTLPPSPEEMTKNQVSLSCANKGFYPSDIAVEWESNGQPENNYKT
TPPVLDSDGSFFLVSRLTVDKSRWQEGNVFSCSVMHEALHNHYTQKSLSLSLGKPAGGGGSEVQLVESGGGLVQPGG
SLRLSCAASGFTFSSYAMSWVRQTPGKGLEWVAVIDSNGGSTYYPDTVKDRFTISRDNSKNTLYLQMNSLRAEDTAVY
YCSSYTNLGAYWGQGTLVTVSAGGGGSGGGGSGGGGSDIQMTQSPSSLSASVGDRVTITCRASQDISGYLNWLQQK
PGGAIKRLIYTTSTLDSGVPKRFSGSGSGTDFTLTISSLQSEDFATYYCLQYASSPFTFGGGTKVEIK*
SEQ ID NO: 231
A PARK PROTEIN, CXCL9-CXCL9-CXCL10-CXCL10-FCKNOB-MAFAP AS SHOWN IN FIG. 5B, IS A
RECOMBINANT PROTEIN COMPRISING PRO-CXCL9-CXCL9-CXCL10-CXCL10-FCKNOB-MAFAP
SEQUENCES.
MKISVAAIPFFLLITIALGTKTESSSRTPVVRKGRCSCISTNQGTIHLQSLKDLKQFAPSPSCEKIEIIATLKNGVQTCLNPDS
ADVKELIKKWEKQVSQKKKQKNGKKHQKKKVLKVRKSQRSRQKKTTGGGTKTESSSRTPVVRKGRCSCISTNQGTIHL
QSLKDLKQFAPSPSCEKIEIIATLKNGVQTCLNPDSADVKELIKKWEKQVSQKKKQKNGKKHQKKKVLKVRKSQRSRQKK
TTGGGTKTESSSRVPLSRTVRCTCISISNQPVNPRSLEKLEIIPASQFCPRVEIIATMKKKGEKRCLNPESKAIKNLLKAVSKE
RSKRSPGGGTKTESSSRVPLSRTVRCTCISISNQPVNPRSLEKLEIIPASQFCPRVEIIATMKKKGEKRCLNPESKAIKNLLKA
VSKERSKRSPGAPGGEPKSSDKTHTCPPCPAPELLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQVYTLPPCPEEM
TKNQVSLWCLVKGFYPSDIAVEWESNGQPENNYKTTPPVLDSDGSFFLYSRLTVDKSRWQEGNVFSCSVMHEALHNH
YTQKSLSLSLGKPAGGGGSQVQLKQSGAELVKPGASVKLSCKTSGYTFTENIIHWVKQRSGQGLEWIGWFHPGSGSIK
YNEKKDKATLTADKSSSTVYMELSRLTSEDSAVYFCARHGGTGRGAMDYWGQGTSVTVSSGGGGSGGGGSGGGGS
DILMTQSPASSVVSLSGQRATISCRASKSVSTSAYSYMHWYQQKPGQPPKWYLASNLESGVPPRFSGSGSGTDFTLNI
HPVEEEDAATYYCQHSRELPYTFGGGTKLEIK
SEQ ID NO: 232
A PARK PROTEIN, CXCL9-CXCL9-CXCL10-CXCL10-FCHOLE-αPAI-1 AS SHOWN IN FIG. 5B, IS A
RECOMBINANT PROTEIN COMPRISING PRO-CXCL9-CXCL9-CXCL10-CXCL10-FCHOLE-αPAI-1 SEQUENCES.
MKISVAAIPFFLLITIALGTKTESSSRTPVVRKGRCSCISTNQGTIHLQSLKDLKQFAPSPSCEKIEIIATLKNGVQTCLNPDS
ADVKELIKKWEKQVSQKKKQKNGKKHQKKKVLKVRKSQRSRQKKTTGGGTKTESSSRTPVVRKGRCSCISTNQGT1HL
QSLKDLKQFAPSPSCEKIEIIATLKNGVQTCLNPDSADVKELIKKWEKQVSQKKKQKNGKKHQKKKVLKVRKSQRSRQKK
TTGGGTKTESSSRVPLSRTVRCTCISISNQPVNPRSLEKLEIIPASQFCPRVEIIATMKKKGEKRCLNPESKAIKNLLKAVSKE
RSKRSPGGGTKTESSSRVPLSRTVRCTCISISNQPVNPRSLEKLEIIPASQFCPRVEIIATMKKKGEKRCLNPESKAIKNLLKA
VSKERSKRSPGAPGGEPKSSDKTHTCPPCPAPELLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQVCTLPPSPEEM
TKNQVSLSCAGFYPSDIAVEWESNGQPENNYKTTPPVLDSDGSFFLVSRLTVDKSRWQEGNVFSCSVMHEALHNH
YTQKSLSLSLGKPAGGGGSQVQLVQSGAEVKKPGASVKVSCKASGYTFTNYGMNWVRQAPGQGLEWMGWINTYTG
EPTYTDDFKGRFTMTLDTSISTAYMELSRLRSDDTAVYYCAKDVSGFVFDYWGQGTLVTVSSGGGGSGGGGSGGGGS
DIVMTQSPDSLAVSLGERATINCKSSQSLLNIIKQKNCLAWYQQKPGQPPKLLIYWASTRESGVPDRFSGSGSGTDFTLTI
SSLQAEDVAVYYCQQYYSYPYTFGQGTKLEIK
SEQ ID NO: 241
FAPSCFV-FCBS1: KNOB
MKWVTFISLLFLFSSAYSQVQLKQSGAELVKPGASVKLSCKTSGYTFTENIIHWVKQRSGQGLEWIGWFHPGSGSIKYN
EKKDKATLTADKSSSTVYMELSRLTSEDSAVYFCARHGGTGRGAMDYWGQGTSVTVSSGGGGSGGGGSGGGGSDIL
MTQSPASSVVSLSGQRATISCRASKSVSTSAYSYMHWYQQKPGQPPKLLIYLASNLESGVPPRFSGSGSGTDFTLNIHP
VEEEDAATYYCαFGGGTKLEIKGAPGGGSGEPKSCDKTHTCPPCPAPELLGGPSVFLFPPKPKDTLMISRTP
EVTαVVVDVSQEDPEVQFNWYVDGVEVHNAKTKPREEQFNSTYRVVSVLTVLHQDWLNGKEYKαKVSNKGLPSSIEK
TISKAKGQPREPQVYTLPPSPEEMTKNQVSLYαLVKGFYPSDIAVEWESNGQPENNYKTTPPVLDSDGSFFLαSRLTVDK
SRWQEGNVFSαSVMHEALHNHYTQKSLSLSLGK**
SEQ ID NO: 251
CCL14(X3)-FCBS2: HOLE
MKISVAAIPFFLLITIALGTKTESSSRGPYHPSECCFTYTTYKIPRQRIMDYYETNSQCSKPGIVFITKRGHSVCTNPSDKWV
QDYIKDMKENGGGTKTESSSRGPYHPSECCFTYTTYKIPRQRIMDYYETNSQCSKPGIVFITKRGHSVCTNPSDKWVQD
YIKDMKENGGGTKTESSSRGPYHPSECCFTYTTYKIPRQRIMDYYETNSQCSKPGIVFITKRGHSVCTNPCSKWVQDYIK
DMKENGAPGGGSSGEPKSCDKTHTCPPCPAPELLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQVYTLPPSPEEM
TKNQVSLTCLVKGFYPSDIAVEWESNGQPENNYKTTPPVLDSDGSFFLTSRLTVDKSRWQEGNVFSCSVMHEALHNHY
TQKSLSLSLGK**
SEQ ID NO: 261
CCL14-CCL5-XCL1-FCBS2: HOLE
MKISVAAIPFFLLITIALGTKTESSSRGPYHPSECCFTYTTYKIPRQRIMDYYETNSQCSKPGIVFITKRGHSVCTNPSDKWV
QDYIKDMKENGGGTKTESSSRSPYSSDTTPCCFAYIARPLPRAHIKEYFYTSGKCSNPAVVFVTRKNRQVCANPEKKWV
REYINSLEMSGGGTKTESSSRVGSEVSDKRTCVSLTTQRLPVSRIKTYTITEGSLRAVIFITKRGLKVCADPQATWVRDVV
RSMDRKSNTRNNMIQTKPTGTQQSTNTAVTLTGGAPGGGSGEPKSCDKTHTCPPCPAPELLGGPSVFLFPPKPKDTL
MISRTPEVTCVVVDVSQEDPEVQFNWYVDGVEVHNAKTKPREEQFNSTYRVVSVLTVLHQDWLNGKEYKCKVSNKG
LPSSIEKTISKAKGQPREPQVYTLPPSPEEMTKNQVSLTCLVKGFYPSDIAVEWESNGQPENNYKTTPPVLDSDGSFFLTS
RLTVDKSRWQEGNVFSCSVMHEALHNHYTQKSLSLSLGK**
SEQ ID NO: 271
CCL14-CCL5-CCL21-CXCL10-FCBS2: HOLE
MKISVAAIPFFLLITIALGTKTESSSRGPYHPSECCFTYTTYKIPRQRIMDYYETNSQCSKPGIVFITKRGHSVCTNPSDKWV
QDYIKDMKENGGGTKTESSSRSPYSSDTTPCCFAYIARPLPRAHIKEYFYTSGKCSNPAVVFVTRKNRQVCANPEKKWV
REYINSLEMGGGTKTESSSRSDGGAQDCCLKYSQRKIPAKVVRSYRKQEPSLGCSIPAILFLPRKRSQAELCADPKELWV
QQLMQHLDKTPSPQKPAQGCRKDRGASKTGKKGKGSKGCKRTERSQTPKGPGGGTKTESSSRVPLSRTVRCTCISISN
QPVNPRSLEKLEIIPASQFCPRVEIIATMKKKGEKRCLNPESKAIKNLLKAVSKERSKRSPGAPGGGSGEPKSCDKTHTCPP
CPAPELLGGPSVFLFPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSPEEMTKNQVSLTCLVKGFYPSDIAVEWESNGQ
PENNYKTTPPVLDSDGSFFLTSRLTVDKSRWQEGNVFSCSVMHEALHNHYTQKSLSLSLGK**

What is claimed is:

1. A protein comprising:
a prochemokine moiety comprising a propeptide moiety fused to a chemokine moiety, wherein the chemokine moiety comprises a N-terminus and a C-terminus, and wherein the propeptide moiety is fused to either the N-terminus or the C-terminus of the chemokine moiety; and
a targeting moiety linked to the prochemokine moiety, wherein the targeting moiety has a binding specificity to a tumor, fibrosis or Alzheimer's Disease associated antigen or receptor.

2. The protein of claim 1, wherein the prochemokine moiety further comprises a leader fused to the propeptide moiety at one end and to chemokine moiety at the opposite end.

3. The protein of claim 1, wherein the protein contains a single prochemokine moiety.

4. The protein of claim 1, wherein the protein comprises at least two prochemokine moieties.

5. The protein of claim 1, wherein at least two prochemokine moieties are linked in tandem through a spacer.

6. The protein of claim 5, wherein the spacer comprises at least one glycine residue.

7. The protein of claim 5, wherein the spacer comprises at least two glycine residues.

8. The protein of claim 1, wherein further comprising an amino acid sequence fused at the C-terminus of the chemokine moiety, wherein the amino acid sequence comprises the mucin region of fractalkine.

9. The protein of claim 1, wherein the chemokine moiety comprises a chemokine amino acid sequence having at least 90% similarity to CC, CXC, CX3C, or C family chemokines.

10. The protein of claim 9, wherein the chemokine moiety comprises a chemokine amino acid sequence having at least 90% similarity to CCL1, CCL3, CCL5, CCL7, CCL14, CCL16, CCL19, CCL20, CCL21, CXCL8, CXCL9, CXCL10, CXCL12, CXCL16, XCL1, or CX3CL1.

11. The protein of claim 1, wherein the chemokine moiety comprises a chemokine amino acid sequence having at least 90% similarity to a chemokine capable of recruiting cells with anti-tumor, anti-fibrosis or anti-Alzheimer's Disease activity.

12. The protein of claim 1, wherein the targeting moiety comprises an antibody, a single chain Fv (scFv) domain, a single variable domain or a natural ligand domain that has a binding specificity to a tumor, fibrosis or Alzheimer's Disease associated antigen or receptor.

13. The protein of claim 1, wherein the targeting moiety has a binding affinity to a tumor cell, tumor stroma antigen, myofibroblast antigen, amyloid or tau protein.

14. The protein of claim 1, further comprising a protein tag covalently linked to at least on end of the protein.

15. The protein of claim 14, wherein the protein tag comprises an amino acid sequence having at least 90% similarity to 8-10 histidines, streptag-2 or immunoglobulin Fc.

16. The protein of claim 1, further comprising an immunoglobulin (IgG) Fc domain.

17. The protein of claim 16, wherein the targeting domain comprises a scFv domain.

18. The protein of claim 16, comprising one scFv domain and one prochemokine moiety.

19. The protein of claim 16, comprising at least two scFv domains.

20. The protein of claim 16, comprising at least two prochemokine moieties.

21. The protein of claim 16, wherein both the prochemokine moiety and the targeting moiety are fused to the IgG Fc domain.

22. The protein of claim 16, wherein the targeting moiety is fused onto one end of the prochemokine moiety and the IgG Fc is fused onto the opposite end of the prohemokine moiety.

23. The protein of claim 16, wherein the IgG Fc domain comprises two immunoglobulin CH2-CH3 domains.

24. The protein of claim 23, wherein one of the immunoglobulin CH2-CH3 domain is fused to the targeting moiety and the other immunoglobulin CH2-CH3 domain is fused to the prochemokine moiety.

25. The protein of claim 1, wherein the protein is an antibody.

26. An isolated nucleic acid encoding the protein of one of claim 1.

27. An expression vector comprising the isolated nucleic acid of claim 30.

28. A host cell comprising the isolated nucleic acid of claim 30 or the expression vector of claim 31.

29. A pharmaceutical composition, comprising the protein of claim 1 and a pharmaceutically acceptable carrier.

30. The pharmaceutical composition of claim 29, further comprising radioisotope, radionuclide, a toxin, a therapeutic agent, a chemotherapeutic agent or a combination thereof.

31. A method of treating a subject with a cancer, fibrosis or Alzheimer's Disease, comprising administering to the subject an effective amount of the protein of claim 1.

32. The method of claim 31, wherein the cancer is capable of expressing one or more tumor associated proteases, wherein the tumor associated protease(s) is capable of cleaving the prochemokine domain off the protein and releasing an active chemokine, and wherein the active chemokine is capable of recruiting immune cells to stimulate therapeutic activity in immune or other cell types within a tumor, fibrosis or Alzheimer's Disease lesion.

33. The method of claim 31, further comprising co-administering an effective amount of a therapeutic agent, wherein the therapeutic agent comprises an antibody, a chemotherapy agent, an enzyme, an anti-estrogen agent, a receptor tyrosine inhibitor, an anti-fibrotic drug, an anti-Alzheimer's Disease drug or a combination thereof.


SEQUENCE LISTING (33 sequences)

SEQ ID NO: 1 - protein, 59 aa, Artificial Sequence
  Synthesized
MKISVAAIPFFLLITIALGTKTESSSRGPYHPSECCFTYTTYKIPRQRIMDYYETNSQC

SEQ ID NO: 2 - protein, 19 aa, Artificial Sequence
  Synthesized
MKISVAAIPFFLLITIALG

SEQ ID NO: 3 - protein, 8 aa, Artificial Sequence
  Synthesized
TKTESSSR

SEQ ID NO: 4 - protein, 59 aa, Artificial Sequence; identical to SEQ ID NO: 1
  Synthesized

SEQ ID NO: 5 - protein, 91 aa, Artificial Sequence
  Synthesized
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 6 - protein, 23 aa, Artificial Sequence
  Synthesized
MKVSAAALAVILIATALCAPASA

SEQ ID NO: 7 - protein, 68 aa, Artificial Sequence
  Synthesized
SPYSSDTTPCCFAYIARPLPRAHIKEYFYTSGKCSNPAVVFVTRKNRQVCANPEKKWVREYINSLEMS

SEQ ID NO: 8 - protein, 95 aa, Artificial Sequence
  Synthesized
MKISVAAIPFFLLITIALGTKTESSSRSPYSSDTTPCCFAYIARPLPRAHIKEYFYTSGKCSNPAVVFVTRKNRQVCANPEKKWVREYINSLEMS

SEQ ID NO: 9 - protein, 73 aa, Artificial Sequence
  Synthesized
KSMQVPFSRCCFSFAEQEIPLRAILCYRNTSSICSNEGLIFKLKRGKEACALDTVGWVQRHRKMLRHCPSKRK

SEQ ID NO: 10 - protein, 76 aa, Artificial Sequence
  Synthesized
QPDAINAPVTCCYNFTNRKISVQRLASYRRITSSKCPKEAVIFKTIVAKEICADPKQKWVQDSMDHLDKQTQTPKT

SEQ ID NO: 11 - protein, 69 aa, Artificial Sequence
  Synthesized
SLAADTPTACCFSYTSRQIPQNFIADYFETSSQCSKPGVIFLTKRSRQVCADPSEEWVQKYVSDLELSA

SEQ ID NO: 12 - protein, 70 aa, Artificial Sequence
  Synthesized
ASNFDCCLGYTDRILHPKFIVGFTRQLANEGCDINAIIFHTKKKLSVCANPKQTWVKYIVRLLSKKVKNM

SEQ ID NO: 13 - protein, 111 aa, Artificial Sequence
  Synthesized
SDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 14 - protein, 103 aa, Artificial Sequence
  Synthesized
TPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 15 - protein, 77 aa, Artificial Sequence
  Synthesized
VPLSRTVRCTCISISNQPVNPRSLEKLEIIPASQFCPRVEIIATMKKKGEKRCLNPESKAIKNLLKAVSKERSKRSP

SEQ ID NO: 16 - protein, 75 aa, Artificial Sequence
  Synthesized
SVTGSCYCGKRISSDSPPSVQFMNRLRKHLRAYHRCLYYTRFQLLSWSVCGGNKDPWVQELMSCLDLKECGHAYS

SEQ ID NO: 17 - protein, 93 aa, Artificial Sequence
  Synthesized
VGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 18 - protein, 76 aa, Artificial Sequence
  Synthesized
QHHGVTKCNITCSKMTSKIPVALLIHYQQNQASCGKRAIILETRQHRLFCADPKEQWVKDAMQHLDRQAAALTRNG

SEQ ID NO: 19 - protein, 100 aa, Artificial Sequence
  Synthesized
MKISVAAIPFFLLITIALGTKTESSSRKSMQVPFSRCCFSFAEQEIPLRAILCYRNTSSICSNEGLIFKLKRGKEACALDTVGWVQRHRKMLRHCPSKRK

SEQ ID NO: 20 - protein, 103 aa, Artificial Sequence
  Synthesized
MKISVAAIPFFLLITIALGTKTESSSRQPDAINAPVTCCYNFTNRKISVQRLASYRRITSSKCPKEAVIFKTIVAKEICADPKQKWVQDSMDHLDKQTQTPKT

SEQ ID NO: 21 - protein, 96 aa, Artificial Sequence
  Synthesized
MKISVAAIPFFLLITIALGTKTESSSRSLAADTPTACCFSYTSRQIPQNFIADYFETSSQCSKPGVIFLTKRSRQVCADPSEEWVQKYVSDLELSA

SEQ ID NO: 22 - protein, 97 aa, Artificial Sequence
  Synthesized
MKISVAAIPFFLLITIALGTKTESSSRASNFDCCLGYTDRILHPKFIVGFTRQLANEGCDINAIIFHTKKKLSVCANPKQTWVKYIVRLLSKKVKNM

SEQ ID NO: 23 - protein, 138 aa, Artificial Sequence
  Synthesized
MKISVAAIPFFLLITIALGTKTESSSRSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 24 - protein, 130 aa, Artificial Sequence
  Synthesized
MKISVAAIPFFLLITIALGTKTESSSRTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 25 - protein, 104 aa, Artificial Sequence
  Synthesized
MKISVAAIPFFLLITIALGTKTESSSRVPLSRTVRCTCISISNQPVNPRSLEKLEIIPASQFCPRVEIIATMKKKGEKRCLNPESKAIKNLLKAVSKERSKRSP

SEQ ID NO: 26 - protein, 102 aa, Artificial Sequence
  Synthesized
MKISVAAIPFFLLITIALGTKTESSSRSVTGSCYCGKRISSDSPPSVQFMNRLRKHLRAYHRCLYYTRFQLLSWSVCGGNKDPWVQELMSCLDLKECGHAYS

SEQ ID NO: 27 - protein, 120 aa, Artificial Sequence
  Synthesized
MKISVAAIPFFLLITIALGTKTESSSRVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 28 - protein, 103 aa, Artificial Sequence
  Synthesized
MKISVAAIPFFLLITIALGTKTESSSRQHHGVTKCNITCSKMTSKIPVALLIHYQQNQASCGKRAIILETRQHRLFCADPKEQWVKDAMQHLDRQAAALTRNG

SEQ ID NO: 29 - protein, 244 aa, Artificial Sequence
  Synthesized
MKISVAAIPFFLLITIALGTKTESSSRGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGPGGGTKTESSSRGVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 30 - protein, 941 aa, Artificial Sequence
  Synthesized
MKISVAAIPFFLLITIALGTKTESSSRGVPLSRTVRCTCISISNQPVNPRSLEKLEIIPASQFCPRVEIIATMKKKGEKRCLNPESKAIKNLLKAVSKERSKRSPGGGTKTESSSRSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGPGGGTKTESSSRSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGPGGGTKTESSSRVGSEVSDKRTCVSLTTQRLPVSRIKTYTITEGSLRAVIFITKRGLKVCADPQATWVRDVVRSMDRKSNTRNNMIQTKPTGTQQSTNTAVTLTGGAPGGEPKSSDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCPEEMTKNQVSLWCLVKGFYPSDIAVEWESNGQPENNYKTTPPVLDSDGSFFLYSRLTVDKSRWQEGNVFSCSVMHEALHNHYTQKSLSLSLGKPAGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 31 - protein, 935 aa, Artificial Sequence
  Synthesized
MKISVAAIPFFLLITIALGTKTESSSRGVPLSRTVRCTCISISNQPVNPRSLEKLEIIPASQFCPRVEIIATMKKKGEKRCLNPESKAIKNLLKAVSKERSKRSPGGGTKTESSSRSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGPGGGTKTESSSRSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGPGGGTKTESSSRVGSEVSDKRTCVSLTTQRLPVSRIKTYTITEGSLRAVIFITKRGLKVCADPQATWVRDVVRSMDRKSNTRNNMIQTKPTGTQQSTNTAVTLTGGAPGGEPKSSDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSPEEMTKNQVSLSCAVKGFYPSDIAVEWESNGQPENNYKTTPPVLDSDGSFFLVSRLTVDKSRWQEGNVFSCSVMHEALHNHYTQKSLSLSLGKPAGGGGSEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIK

SEQ ID NO: 32 - protein, 910 aa, Artificial Sequence
  Synthesized
MKISVAAIPFFLLITIALGTKTESSSRTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTTGGGTKTESSSRTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTTGGGTKTESSSRVPLSRTVRCTCISISNQPVNPRSLEKLEIIPASQFCPRVEIIATMKKKGEKRCLNPESKAIKNLLKAVSKERSKRSPGGGTKTESSSRVPLSRTVRCTCISISNQPVNPRSLEKLEIIPASQFCPRVEIIATMKKKGEKRCLNPESKAIKNLLKAVSKERSKRSPGAPGGEPKSSDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCPEEMTKNQVSLWCLVKGFYPSDIAVEWESNGQPENNYKTTPPVLDSDGSFFLYSRLTVDKSRWQEGNVFSCSVMHEALHNHYTQKSLSLSLGKPAGGGGSQVQLKQSGAELVKPGASVKLSCKTSGYTFTENIIHWVKQRSGQGLEWIGWFHPGSGSIKYNEKKDKATLTADKSSSTVYMELSRLTSEDSAVYFCARHGGTGRGAMDYWGQGTSVTVSSGGGGSGGGGSGGGGSDILMTQSPASSVVSLSGQRATISCRASKSVSTSAYSYMHWYQQKPGQPPKLLIYLASNLESGVPPRFSGSGSGTDFTLNIHPVEEEDAATYYCQHSRELPYTFGGGTKLEIK

SEQ ID NO: 33 - protein, 910 aa, Artificial Sequence
  Synthesized
MKISVAAIPFFLLITIALGTKTESSSRTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTTGGGTKTESSSRTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTTGGGTKTESSSRVPLSRTVRCTCISISNQPVNPRSLEKLEIIPASQFCPRVEIIATMKKKGEKRCLNPESKAIKNLLKAVSKERSKRSPGGGTKTESSSRVPLSRTVRCTCISISNQPVNPRSLEKLEIIPASQFCPRVEIIATMKKKGEKRCLNPESKAIKNLLKAVSKERSKRSPGAPGGEPKSSDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSPEEMTKNQVSLSCAVKGFYPSDIAVEWESNGQPENNYKTTPPVLDSDGSFFLVSRLTVDKSRWQEGNVFSCSVMHEALHNHYTQKSLSLSLGKPAGGGGSQVQLVQSGAEVKKPGASVKVSCKASGYTFTNYGMNWVRQAPGQGLEWMGWINTYTGEPTYTDDFKGRFTMTLDTSISTAYMELSRLRSDDTAVYYCAKDVSGFVFDYWGQGTLVTVSSGGGGSGGGGSGGGGSDIVMTQSPDSLAVSLGERATINCKSSQSLLNIIKQKNCLAWYQQKPGQPPKLLIYWASTRESGVPDRFSGSGSGTDFTLTISSLQAEDVAVYYCQQYYSYPYTFGQGTKLEIK